(19) United States
(12) Patent Application Publication
(10) Pub. No.: US 2023/0248621 A1
(43) Pub. Date: Aug. 10, 2023

(54) POWDER/GRANULES FOR MAKING A SHOWER GEL OR SHAMPOO

(71) Applicant: MANN & SCHROEDER GMBH, Siegelsbach (DE)

(72) Inventors: Klaus HEMPEL, Weinhem (DE); Hannah SCHOLZ, Siegelsbach (DE); Christina LEE, Harthausen (DE)

(21) Appl. No.: 18/105,359

(22) Filed: Feb. 3, 2023

(30) Foreign Application Priority Data
Feb. 10, 2022 (DE) 102022103160.8

(51) Int. Cl. A61K 8/02; A61Q 5/02; A61Q 19/10; A61K 8/41

(52) U.S. Cl. CPC A61K 8/0225; A61K 8/416; A61Q 5/02; A61Q 19/10; A61K 2800/596; A61K 2800/652; A61K 2800/882

(57) ABSTRACT

The invention relates to a powder/granular material for producing a shower gel or shampoo, which contains a salt of a mono-C₈₋₁₈-alkyl sulfate, in particular a salt of coco sulfate or lauryl sulfate, and an ammonium salt. The invention furthermore also relates to the use of the powder/granular material according to the invention for producing a shower gel or shampoo. The invention moreover relates to a shower gel/shampoo, which contains a salt of a mono-C₈₋₁₈-alkyl sulfate, in particular a salt of coco sulfate or lauryl sulfate, an ammonium salt and water, characterized in that a further cosurfactant is present at most in a quantity by weight of less than half the quantity by weight of the salt of a mono-C₈₋₁₈-alkyl sulfate.

The present invention relates to a powder/granular material for producing a shower gel or shampoo, which contains a salt of a mono-C₈₋₁₈-alkyl sulfate, in particular a salt of coco sulfate or lauryl sulfate, and an ammonium salt. The present invention furthermore also relates to the use of the powder/granular material according to the invention for producing a shower gel or shampoo. The present invention moreover relates to a shower gel/shampoo.

Conventional liquid shampoos and shower gels are normally sold in plastic packaging, which generates a considerable amount of plastic waste after the shampoos and shower gels have been used up.

In order to reduce the amount of plastic waste, a powder or granular material can for example be provided, which can be repeatedly added to the same container and, when mixed with water, produces a shampoo or shower gel. In this way, a sustainable product can be provided, with which packaging and transport costs can be saved.

The components must be present in powder or granular form, with the result that it is necessary here to rely on surfactants which are available in solid form and are highly soluble in tepid water.

Here, for example, a salt of a mono-C₈₋₁₈-alkyl sulfate, in particular a salt of coco sulfate or lauryl sulfate, is suitable, as it can be obtained as a solid and is often used in common natural cosmetic shower gels and shampoos.

When a salt of a mono-C₈₋₁₈-alkyl sulfate, in particular a salt of coco sulfate or lauryl sulfate, is used in shampoos or shower gels, a further cosurfactant, such as for example a salt of coco glutamate, is usually used in order to prevent the salt of a mono-C₈₋₁₈-alkyl sulfate from crystallizing out with cations/salts contained in the water or in other raw materials. As almost all known cosurfactants are liquid, they cannot be used or can only be used to a very limited extent for a powder or granular material. If, however, a cosurfactant is not used, and the salt of the mono-C₈₋₁₈-alkyl sulfate is used as main surfactant, approximately one to two days after dissolution in warm tap water a white, creamy suspension forms from the hitherto clear shower gel or shampoo. This effect is intensified or accelerated by the Ca and Mg ions contained in the (hard) tap water, with the result that the resulting shower gel or shampoo does not remain a clear product for a period of more than one to two days.

Normally, xanthan is added to a granular material which is subsequently to be dissolved in tap water so that the resulting solution achieves a certain viscosity and is thus stabilized. However, this results in a product which has an undesirably gelatinous structure and is usually not clear. It is therefore desirable to provide a product which has an improved performance with respect to transparency and flow behavior.

The object of the present invention is therefore to provide a powder or granular material which, when mixed with water, preferably tap water, produces a clear, flowable and viscous shower gel or shampoo which remains stable for a period of several weeks, i.e. in which the surfactant does not crystallize out within a few weeks or days. It is desirable not to have to add a further cosurfactant since most cosurfactants are present in liquid or dissolved form, with the result that a dry powder or granular material cannot be provided.

The named object is achieved by a powder or granular material as provided herein. The powder/granular material according to the invention is used to produce a shower gel or shampoo and contains a salt of a mono-C₈₋₁₈-alkyl sulfate, in particular a salt of coco sulfate or lauryl sulfate, and an ammonium salt. Surprisingly, the inventors of the present invention have found that by adding an ammonium salt to the salt of a mono-C₈₋₁₈-alkyl sulfate a further addition of a cosurfactant is not necessary in order to obtain a clear product for a number of weeks after dissolution of the powder or granular material. A further advantage of the use of the ammonium salt is that it has thickening properties, with the result that the consistency usual for shower gels and shampoos is obtained.

An alkali metal mono-C₈₋₁₈-alkyl sulfate or ammonium mono-C₈₋₁₈-alkyl sulfate is preferably used as salt of a mono-C₈₋₁₈-alkyl sulfate in the powder or granular material according to the invention, wherein ammonium, sodium or potassium mono-C₈₋₁₈-alkyl sulfate is preferred. The use of the last-named salts has the advantage compared with other alkali metals that they are inexpensive and easily water-soluble, wherein sodium mono-C₈₋₁₈-alkyl sulfate is more preferred. According to the invention, the salt of a mono-C₈₋₁₈-alkyl sulfate can also be a mixture of different salts of one or more different C₈₋₁₈-alkyl sulfates, i.e. different C₈₋₁₈-alkyl sulfates with different C chain lengths, as in the case of e.g. coco sulfate.

The mono-C₈₋₁₈-alkyl sulfate is preferably a mono-C₁₂₋₁₈-alkyl sulfate, in particular lauryl sulfate or coco sulfate.

The salt of a mono-C₈₋₁₈-alkyl sulfate is particularly preferably sodium lauryl sulfate or sodium coco sulfate. These have the advantage that they are particularly highly water-soluble.

By a salt of a coco sulfate is meant herein a salt of a mono-C₁₂₋₁₈-alkyl sulfate, or a mixture of a variety of these alkyl sulfates. The salt of the coco sulfate is usually obtained through a reaction of coconut oil or palm oil with sulfuric acid and a subsequent reaction with a salt.

In the powder or granular material according to the invention, the ammonium salt can be an ammonium halide, ammonium sulfate or ammonium carbonate, wherein an ammonium halide is preferred. According to the invention, preferred ammonium halides are ammonium chloride or bromide, wherein ammonium chloride is more preferred.

The ammonium ion of the ammonium salt can be an organic ammonium compound, in particular a mono-, di- or tri-alkylated ammonium ion, in particular triethylammonium. The ammonium ion is preferably an NH₄⁺ ion, however. A particularly good solubility can thereby be guaranteed.

In a further design according to the invention, the powder or granular material can additionally contain an organic carboxylic acid. Here, it is preferred for the organic carboxylic acid to have a plurality of carboxylic acid groups. The organic carboxylic acid particularly preferably has two, three or four carboxylic acid groups, wherein an organic carboxylic acid having three carboxylic acid groups is preferred. The organic carboxylic acid can preferably also have one or more hydroxyl groups. Examples of organic carboxylic acids that can be used are citric acid or lactic acid. The organic carboxylic acid is preferably present in a quantity of from 2.5 wt.-% to 15 wt.-% in the powder or granular material, relative to the total weight of the powder/granular material. The use of organic acids makes it possible to set the pH, in order to set the pH of the resulting shower gel or shampoo in a range of less than 5, but preferably not less than 2. This has the advantage that, when a preservative such as for example benzoic acid is used, it is present as a free acid in a form that is adequate for preservation.

In a further design according to the invention, the powder/granular material preferably contains 40 wt.-% to 90 wt.-%, more preferably 50 wt.-% to 80 wt.-%, and most preferably 60 wt.-% to 70 wt.-% of the salt of a mono-C₈₋₁₈-alkyl sulfate, in particular of the salt of coco sulfate or lauryl sulfate, in each case relative to the total weight of the powder/granular material. In the case of a quantity that is too large, the salt of a mono-C₈₋₁₈-alkyl sulfate, in particular the salt of coco sulfate or lauryl sulfate, does not dissolve adequately in water. In the case of a quantity that is too small, the shower gel or shampoo resulting therefrom does not have adequate washing properties.

In a further design according to the invention, the powder/granular material preferably contains 10 wt.-% to 40 wt.-%, more preferably 12 wt.-% to 30 wt.-% and most preferably 15 wt.-% to 25 wt.-% of the ammonium salt, in each case relative to the total weight of the powder/granular material. In the case of a quantity that is too large, the resulting shower gel or shampoo is too viscous. In the case of a quantity that is too small, the salt of a mono-C₈₋₁₈-alkyl sulfate, in particular the salt of coco sulfate or lauryl sulfate, crystallizes out after a few days.

According to a further preferred design according to the invention, the powder/granular material contains a molar excess of ammonium ions relative to mono-C₈₋₁₈-alkyl sulfate ions, in particular coco sulfate ions or lauryl sulfate ions, in particular a molar excess in the range of from 20 to 1 to 1.1 to 1, more preferably 10 to 1 to 1.3 to 1, and even more preferably 5 to 1 to 1.5 to 1. Such an excess ensures a good solubility of the powder/granular material and prevents a turbidity of the shower gel/shampoo produced therefrom. In the case of a quantity of ammonium ions that is too small, a clear mixture cannot be obtained.

If the powder/granular material according to the invention contains a further cosurfactant, this is present at most in a quantity by weight of less than half the quantity by weight of the salt of a mono-C₈₋₁₈-alkyl sulfate, in particular of the salt of coco sulfate or lauryl sulfate, relative to the total weight of the powder/granular material. The advantage in this respect, namely using at most small quantities of further cosurfactants, is that, through the use of the ammonium salt, the salt of a mono-C₈₋₁₈-alkyl sulfate, in particular the salt of coco sulfate or lauryl sulfate, can be prevented from precipitating. It is particularly preferred according to the invention if the powder/granular material according to the invention does not contain a further (liquid) cosurfactant.

In addition to the constituents previously named, the powder/granular material according to the invention can also contain further constituents such as e.g. preservatives or fragrances. Examples of preservatives are sodium benzoate and potassium sorbate. One example of a fragrance is the perfume oil Blue Care 9070211. It is preferred according to the invention for preservatives to be used in a quantity of from 0 wt.-% to 8 wt.-%, more preferably in a quantity of from 0 wt.-% to 5 wt.-%, relative to the total weight of the powder/granular material. It is furthermore preferred for the fragrance to be used in a quantity of from 0 wt.-% to 8 wt.-%, more preferably in a quantity of from 0 wt.-% to 5 wt.-%, relative to the total weight of the powder/granular material.

The present invention also relates to a powder/granular material which consists of the following constituents:
40 wt.-% to 90 wt.-%, more preferably 50 wt.-% to 80 wt.-%, and most preferably 60 wt.-% to 70 wt.-% of a salt of a mono-C₈₋₁₈-alkyl sulfate, in particular of a salt of coco sulfate or lauryl sulfate;
10 wt.-% to 40 wt.-%, more preferably 12 wt.-% to 30 wt.-%, and most preferably 15 wt.-% to 25 wt.-% of an ammonium salt;
0 wt.-% to 15 wt.-%, more preferably 2.5 wt.-% to 15 wt.-% organic carboxylic acid;
0 wt.-% to 8 wt.-%, more preferably 0 wt.-% to 5 wt.-% preservative; and
0 wt.-% to 8 wt.-%, more preferably 0 wt.-% to 5 wt.-% fragrance, in each case relative to the total weight of the powder/granular material.

A further embodiment of the invention relates to the use of a powder/granular material according to the invention for producing a shower gel or shampoo. For this, the powder/granular material according to the invention is mixed with water, preferably tap water. In other words, the present invention also relates to a method for producing a shower gel or shampoo, in which the powder/granular material according to the invention is mixed with water. It is preferred for water to be added to the powder/granular material in such a quantity that, in the resulting shower gel or shampoo, the weight ratio of powder/granular material to water lies in the range of from 1:4 to 1:20, more preferably in the range of from 1:6 to 1:15 and most preferably in the range 1:8 to 1:10. A quantity of water that is too large results in a product that is too watery with lower washing performance. In the case of a quantity of water that is too small, the product is too viscous for the area of application, or the powder/granular material does not dissolve completely.

The present invention also relates to a shower gel/shampoo which contains a salt of a mono-C₈₋₁₈-alkyl sulfate, in particular a salt of coco sulfate or lauryl sulfate, an ammonium salt and water, which is characterized in that a further cosurfactant is present at most in a quantity by weight of less than half the quantity by weight of the salt of a mono-C₈₋₁₈-alkyl sulfate, in particular of a salt of coco sulfate or lauryl sulfate. Such small quantities of a further cosurfactant can therefore be used because the combination of salt of a mono-C₈₋₁₈-alkyl sulfate, in particular salt of coco sulfate or lauryl sulfate, with an ammonium salt already prevents the salt of a mono-C₈₋₁₈-alkyl sulfate, in particular a salt of coco sulfate or lauryl sulfate, from precipitating for several weeks. It is therefore also possible according to the invention to provide a clear shower gel/shampoo in this way which is stable for weeks and which essentially does not contain a further cosurfactant.

Demineralized or mineral-containing water can be used as water for producing the shower gel or shampoo according to the invention. In particular, however, the powder/granular material according to the invention is to be highly soluble in normal tap water and stable for a period of several weeks.

The shower gel/shampoo according to the invention is particularly preferably one which can be obtained by mixing a powder/granular material according to the invention with water.

According to a further preferred design according to the invention, the shower gel/shampoo preferably has a viscosity in the range of from 2,000 mPa*s to 15,000 mPa*s, and more preferably in the range of from 3,000 mPa*s to 7,000 mPa*s, which is measured according to Brookfield at 20° C. (spindle 05, 20 rpm). In the case of such a viscosity, the shower gel/shampoo can be handled particularly well and has a pleasant feel during use.

According to a further preferred design according to the invention, the shower gel/shampoo preferably has a turbidity of less than 20 NFU, more preferably less than 10 NFU and even more preferably less than 5 NFU, measured according to DIN EN ISO 7027-1. Such a shower gel is particularly clear and transparent and creates a desired optical impression.

In the case of a determination of the flowability at 20° C. in a cone/plate measuring system, the shower gel/shampoo according to the invention can preferably have a yield stress of at least 50 Pa, determined with a variation in the shear stress of from 0.4 to 100 Pa in the shear stress test, and/or have a yield stress of at least 65 Pa, determined with a variation in the shear rate of from 0.1 to 1000 s⁻¹ in the shear rate test. The measurement curve can be generated via a Herschel-Bulkley regression. For example, a Brookfield RCT-50-1 cone/plate measuring system can be used. These parameters reflect the haptic behavior, or the flowability, as well as the spreadability of the shower gel/shampoo. Such a flowability and spreadability is particularly advantageous.

Further, in the case of a thixotropy measurement in the range of from 0.4 to 50 Pa, the shower gel/shampoo according to the invention can have a thixotropic area of less than 10,000 Pa*s, in particular of less than 1,000 Pa*s. Such a low thixotropy guarantees uniform flow properties and thus a uniform spreadability of the shower gel/shampoo.

All preferred features and definitions, which are mentioned herein in connection with the various embodiments and designs, are also to apply to the respective other embodiments and designs herein.

The present invention will now be explained with reference to the following examples:

### EXAMPLE 1: PREPARATION OF A GRANULAR MATERIAL OR SHOWER GEL/SHAMPOO ACCORDING TO THE INVENTION

According to Table 1 below, a granular material according to the invention with the constituents specified therein was prepared. Furthermore, 12 g of the granular material was mixed with 88 g tap water (25° C.) to form a shampoo/shower gel. After complete dissolution, a clear mixture formed which remained unchanged for 3 months in a temperature window in the range of from 5° C. to 40° C. At temperatures around 5° C., crystals temporarily formed, which could be easily dissolved again when the temperature was increased by 10° C. to 15° C., however.

TABLE 1

| Constituent | Name | Name | Granular material (proportions of the constituents) | Shampoo/ shower gel (proportions of the constituents) |
|---|---|---|---|---|
| | | | [wt.-%] | [wt.-%] |
| Solvent | WATER DEMIN. | | | 88 |
| Surfactant anionic | SODIUM COCO SULFATE MB | Sulfopon 1216G; BASF | 65.84 | 7.90 |
| Auxiliary material | AMMONIUM CHLORIDE | Chemsolte 2668; TH Geyer | 20.83 | 2.50 |
| Organic carboxylic acid | CITRIC ACID DAB (GRIES) | Citric acid DAB (Gries); Brenntag GmbH | 7.5 | 0.90 |
| Preservative | SODIUM BENZOATE | Sodium benzoate Prills 25; RFI Food Ingredients | 1.67 | 0.20 |
| Preservative | POTASSIUM SORBATE | Potassium sorbate ZTN; Ter Hell | 0.83 | 0.10 |
| Perfume oil | PO BLUE CARE 9070211 | Perfume Blue Care; Robertet S.A. | 3.33 | 0.40 |

### COMPARISON EXAMPLES 1 TO 4 WITH ALKALI CHLORIDES INSTEAD OF AMMONIUM CHLORIDE

Instead of the quantity of ammonium chloride specified in Table 1, in each case NaCl (comparison example 1), KCI (comparison example 2), CaCl2 (comparison example 3) and MgCl2 (comparison example 4) were used here. After dissolution in demineralized water, analogously to Example 1 according to the invention, it was possible in the case of NaCl, KCI and MgCl₂ to obtain initially clear shower gels/shampoos in each case, in which, however, after one to two days a turbidity due to precipitation of the sodium coco sulfate was observed. In the case of CaCl₂, it was not possible to obtain a clear product through the addition of water from the start.

### COMPARISON EXAMPLES 5 AND 6 WITH OTHER SURFACTANTS INSTEAD OF SODIUM COCO SULFATE:

In further comparison tests, instead of the quantity of sodium coco sulfate specified in Table 1, in each case sodium cocoyl isethionate (comparison example 5) or sodium lauryl sulfoacetate (comparison example 6) was used. The granular material obtained was very poorly soluble in water in both cases and did not result in a clear product.

### COMPARISON EXAMPLE 7

When ammonium coco sulfate was used without a further ammonium salt, the granular material obtained was likewise very poorly soluble in water and did not result in a clear product.

### COMPARISON EXAMPLE 8: POWDER/GRANULAR MATERIAL WITH XANTHAN AS AUXILIARY MATERIAL/STABILIZER INSTEAD OF AMMONIUM SALT:

Here, a powder/granular material with the following ingredients was provided: sodium coco sulfate, xanthan, Theobroma cacao seed butter, citric acid, potassium sorbate, silica gel, sodium benzoate, Butyrospermum parkii butter, Aloe barbadensis leaf juice, Camellia sinensis leaf powder, Cl 75100, Gardenia jasminoides fruit extract, maltodextrin, limonene, benzyl salicylate, eugenol, perfume

### COMPARISON EXAMPLE 9 STANDARD SHAMPOO

The standard shampoo has the following composition:

TABLE 2

Data in percent by weight

| | |
|---|---|
| WATER DEMIN. | 78.0899475 |
| ETHER SULFATE MB | 10.8 |
| Cocamidopropyl betaine | 5.0 |
| GLYCEROL 99.5% | 2.0 |
| DEAD SEA SALT | 1.5 |
| LAMESOFT PO 65 MB | 1.0 |
| WHITE TEA EXTR A KBA 0485621 | 0.1 |
| OLIVE LEAF EXTR GLY 0487304 | 0.01 |
| PO WHITE TEA & MAGNOLIA 68404 | 0.5 |
| SODIUM BENZOATE | 0.4 |
| POTASSIUM SORBATE | 0.2 |
| CITRIC ACID DAB (GRIT) | 0.4 |
| TABLE SALT COARSE (BRINE) | 0.0000025 |
| FD & C BLUE NO.1, C.I.42090 100301 | 0.00005 |

To produce a shampoo/shower gel, 20 g of the powder is rapidly added to 240 ml tepid water and immediately shaken vigorously 10 times. It is then left to stand for 15 minutes and shaken vigorously once again.

### INVESTIGATIONS OF VARIOUS PROPERTIES OF THE COMPOSITIONS OF EXAMPLE 1 AND COMPARISON EXAMPLES 8 AND 9:

### 1. Foaming Behavior

In each case 5 wt.-% aqueous surfactant solutions are prepared from the shampoos/shower gels of Example 1 and comparison examples 8 and 9. In each case 1 g of the surfactant solutions is introduced into a 10 ml graduated cylinder. Each of the surfactant solutions is shaken uniformly for 10 s.

The shampoo/shower gel according to the invention of Example 1 here foams up to the 5 ml mark of the graduated cylinder, whereas the surfactant solution according to comparison example 8 only foams up to the 2 ml mark and the surfactant solution according to comparison example 9 only foams up to the 5 ml mark.

In other words, the shampoo according to the invention according to Example 1 performs better with respect to the foaming behavior than the shampoo according to comparison example 9 and much better than the standard shampoo according to comparison example 8.

Furthermore, the foam stability is investigated after standing for 20 minutes and 60 minutes:

In the case of all shampoos, the foam stability is comparably good, i.e. a decrease in the quantity of foam (fill level of the graduated cylinder) is barely perceptible in this period.

### 2. Turbidity

For the shampoos produced according to Example 1 and comparison example 8, the turbidity is determined according to DIN EN ISO 7027-1. For the shampoo according to Example 1 a value of 4.6 FNU is obtained, and for the shampoo according to comparison example 8 a value of >1000 FNU is obtained, i.e. a value which lies above the measurement range.

For the shampoo according to the invention, this results in the latter having a much lower turbidity, compared with shampoos with xanthan as thickener, which is perceived as pleasant and desirable by the consumer.

### 3. Flowability and Haptic Behavior

To determine the flowability and the haptic behavior of the shampoos from Example 1 and comparison example 8, the thixotropy, which is a measure of the flowability and the haptic behavior of the resulting shampoos, is determined over the variation in the shear stress and the shear rate in a viscosity diagram. The lower the thixotropy, the better the flowability and the haptic behavior of the shampoo.

For this, the following equipment is used:
RSTCPS rheometer, Brookfield
RST-TC-PA thermostat, Brookfield
RCT-50-1 cone/plate measuring system, Brookfield
Rheo 3000 measurement software version 2.1.109.24663, Brookfield

For this, the shear stress is plotted against the shear rate in the respective measurement tests. 300 measuring points are recorded at a constant temperature of 20° C. The test is carried out by double determination. The evaluation is effected visually. The measurement curve is generated via a Hershel-Bulkley regression.

Measurement settings:
variation in the shear stress: 0.4 to 100 Pa
variation in the shear rate: 0.1 to 1000 1/s
thixotropy measurements: 0.4 to 50 Pa

The measurement results revealed a thixotropic area of 513 Pa*s for the shampoo according to the invention of Example 1 and a thixotropic area of 15,639 Pa*s for the shampoo of comparison example 8.

This results in an only slight thixotropic behavior and thus an excellent flow rate and haptic behavior for the shampoo according to the invention. The comparison shampoo on the other hand exhibits a significantly thixotropic behavior and thus substantially lower flow properties and haptic behavior.

1. A powder or granular material for producing a shower gel or shampoo, which contains a salt of a mono-C₈₋₁₈-alkyl sulfate and an ammonium salt.

2. The powder or granular material according to claim 1, in which the salt of the mono-C₈₋₁₈-alkyl sulfate is ammonium mono-C₈₋₁₈-alkyl sulfate or an alkali mono-C₈₋₁₈-alkyl sulfate.

3. The powder or granular material according to claim 1, in which the mono-C₈₋₁₈-alkyl sulfate is a mono-C₁₂₋₁₈-alkyl sulfate .

4. The powder or granular material according to claim 1, in which the ammonium salt is an ammonium halide.

5. The powder or granular material according to claim 1, which contains 40 wt.-% to 90 wt.-% of the salt of a mono-C₈₋₁₈-alkyl sulfate, relative to the total weight of the powder/granular material.

6. The powder or granular material according to claim 1, which contains 10 wt.-% to 40 wt.-% of the ammonium salt, relative to the total weight of the powder/granular material.

7. The powder or granular material according to claim 1, which contains a molar excess of ammonium ions relative to mono-C₈₋₁₈-alkyl sulfate ions, comprising a molar excess ina range of from 20 to 1 to 1.1 to 1, or from 10 to 1 to 1.3 to 1, or from 5 to 1 to 1.5 to 1.

8. The powder or granular material according to claim 1, which contains a further cosurfactant at most in a quantity by weight of less than half the quantity by weight of the salt of a mono-C₈₋₁₈-alkyl sulfate.

9. The powder or granular material according to claim 1, which does not contain a further liquid cosurfactant.

10. Use of a the powder or granular material according to claim 1 for producing a shower gel or shampoo.

11. Use of the powder or granular material according to claim 10, in which the powder or granular material is mixed with water.

12. A shower gel or shampoo, which contains a salt of a mono-C₈₋₁₈-alkyl sulfate an ammonium salt and water, wherein a further cosurfactant is present at most in a quantity by weight of less than half the quantity by weight of the salt of a mono-C₈₋₁₈-alkyl sulfate.

13. The shower gel or shampoo according to claim 12, wherein the shower gel or shampoo excludes a further cosurfactant.

14. The shower gel or shampoo according to claim 12, obtained by mixing a powder or granular material, which contains a salt of a mono-C₈₋₁₈-alkyl sulfate and an ammonium salt, with water.
